(19) United States
(12) Patent Application Publication
(10) Pub. No.: US 2026/0131489 A1
(43) Pub. Date: May 14, 2026

(54) MOTOR PACK, MOUNTING ASSEMBLY, AND MOUNTING ASSEMBLY DETECTION SYSTEM INCLUDING THE SAME

(71) Applicant: LIVSMED INC., Seongnam-si (KR)

(72) Inventors: Young Jae SONG, Seongnam-si (KR); Ha Kyoon KIM, Seongnam-si (KR)

(21) Appl. No.: 19/389,511

(22) Filed: Nov. 14, 2025

(30) Foreign Application Priority Data
Nov. 14, 2024 (KR) 10-2024-0161714

(51) Int. Cl. B25J 19/02; A61B 34/00

(52) U.S. Cl. CPC B25J 19/027; A61B 34/70

(57) ABSTRACT

A mounting assembly detection system includes a motor pack configured to provide a driving force to a surgical instrument, and a mounting assembly configured to be detachably mounted on the motor pack. The motor pack includes a first sensor module disposed adjacent to a mounting part configured to provide an area in which the mounting assembly is mounted. The first sensor module includes a first magnetic sensor and a first magnetic body having a first area adjacent to the first magnetic sensor and a second area adjacent to the mounting part. The mounting assembly includes a detection target module including a first magnet disposed at a position corresponding to the first sensor module.

### CROSS-REFERENCE TO RELATED APPLICATION

This application is based on and claims priority under 35 U.S.C. §119 to Korean Patent Application No. 10-2025-0161714, filed on November 14, 2024, in the Korean Intellectual Property Office, the disclosure of which is incorporated by reference herein in its entirety.

### BACKGROUND

### 1. Field

The disclosure relates to a motor pack, a mounting assembly, and a mounting assembly detection system including the same.

### 2. Description of the Related Art

In medical terms, surgery refers to curing an illness by cutting, incising, or manipulating the skin, mucous membranes, or other tissues by using medical devices. In particular, open surgery, which involves cutting open the skin at the surgical site and treating, shaping, or removing the organs inside, causes problems such as bleeding, side effects, patient pain, and scarring. Therefore, surgery using a robot or surgery performed by forming a small hole in the skin and inserting only a medical device, such as a laparoscope, a surgical instrument, or a microsurgical microscope, has recently attracted attention as an alternative.

Surgical robots refer to robots which have the function of replacing surgical operations performed by surgeons. Such surgical robots have the advantages of being able to perform more accurate and precise motions than humans and perform remote surgeries.

Surgical robots which have been currently developed worldwide include bone surgical robots, laparoscopic surgical robots, stereotactic surgical robots, and the like. Laparoscopic surgical robots are robots which perform minimally invasive surgery using a laparoscope and small surgical tools.

Surgical robots usually include a master robot and a slave robot. When the surgeon manipulates the control lever (e.g., handle) equipped on the master robot, the surgical tool coupled to the robot arm of the slave robot or held by the robot arm is manipulated to perform the surgery.

Laparoscopic surgery is a cutting-edge surgical technique which drills a small hole in a navel area, inserts a laparoscope, which is an endoscope for looking inside the abdomen, into the small hole, and performs surgery. Laparoscopic surgery is a field which is expected to see great development in the future. Recent laparoscopes are capable of obtaining clearer and more magnified images than visual inspection by using computer chips mounted thereon. Furthermore, advances have made it possible to perform any type of surgery by using specially designed laparoscopic surgical instruments while viewing a screen through a monitor.

The aforementioned background technology is technical information possessed by the inventor for derivation of the disclosure or acquired by the inventor during the derivation of the disclosure, and is not necessarily prior art disclosed to the public before the application of the present disclosure.

### SUMMARY

The disclosure provides a mounting assembly detection system capable of detecting a mounting assembly mounted on a motor pack and minimizing damage to a sensor due to contact or pressure from an external object.

In an embodiment, a mounting assembly detection system may include a motor pack configured to provide a driving force to a surgical instrument, and a mounting assembly configured to be detachably mounted on the motor pack, where the motor pack may include at least one sensor module disposed adjacent to a mounting part configured to provide an area in which the mounting assembly is mounted, the at least one sensor module including a first magnetic sensor and a first magnetic body having a first area adjacent to the first magnetic sensor and a second area adjacent to the mounting part, and the mounting assembly may include at least one detection target module including a first magnet disposed at a position corresponding to the first sensor module.

In another embodiment, the first magnet may be disposed at a position corresponding to the second area of the first magnetic body.

In the other embodiment, the mounting assembly detection system may include a first state in which the mounting assembly is mounted on the mounting part and a second state in which the mounting assembly is detached from the mounting part, and the at least one detection target module may be closer to the at least one sensor module in the first state than in the second state.

In the other embodiment, the first magnetic body may be magnetized by the first magnet in the first state, and the first magnetic sensor may be configured to detect the at least one detection target module when the first magnetic body is magnetized.

In the other embodiment, the first magnetic sensor may be spaced apart from the first magnet by a first preset distance while the mounting assembly is in a state of being mounted on the motor pack.

In the other embodiment, the first magnetic body may be a longitudinal member formed with a length corresponding to a first preset distance at which the first magnetic sensor is spaced apart from the first magnet.

In the other embodiment, a the first magnetic body may be spaced apart from the first magnet by a second preset distance while the mounting assembly is in a state of being mounted on the motor pack.

In the other embodiment, the at least one sensor module may further include a sensor housing configured to accommodate the first magnetic sensor and the first magnetic body, and at least a portion of the sensor housing may be coupled to the mounting part.

In the other embodiment, the sensor housing may include a partition wall disposed at one end portion of the first magnetic body, and the partition wall may limit contact between the first magnet and the first magnetic body.

In the other embodiment, the at least one sensor module of the motor pack may include a plurality of sensor modules, and the at least one detection target module of the mounting assembly may include a plurality of detection target modules corresponding to the plurality of sensor modules.

In the other embodiment, the mounting assembly detection system may be configured to detect the mounting assembly when the plurality of first sensor modules detect all of the plurality of detection target modules.

In the other embodiment, the motor pack may further include a second magnetic sensor and a second magnetic body having a first area adjacent to the second magnetic sensor and a second area adjacent to the mounting part, and the mounting assembly may further include a bridge module including a third magnetic body disposed at a position corresponding to the second sensor module.

In the other embodiment, when the mounting assembly is an adapter disposed between the surgical instrument and the motor pack, the third magnetic body and the second magnetic body may be magnetized by a second magnet provided in the surgical instrument while the adapter and the surgical instrument are in a state of being mounted on the motor pack.

In the other embodiment, a mounting assembly detection system may include a motor pack configured to provide a driving force to a surgical instrument, and a mounting assembly configured to be detachably mounted on the motor pack, where the motor pack may include a sensor module disposed adjacent to a mounting part configured to provide an area in which the mounting assembly is mounted, the sensor module including a magnetic sensor, a first magnet which is movable between a position adjacent to the magnetic sensor and a position spaced apart from the magnetic sensor, and a magnet carrier configured to support the first magnet and limit a range of movement of the first magnet, and the mounting assembly may include a detection target module disposed at a position corresponding to the sensor module.

In the other embodiment, the detection target module may include a second magnet or a magnetic body disposed to face the first magnet.

In the other embodiment, the mounting assembly detection system may include a first state in which the mounting assembly is mounted on the mounting part and a second state in which the mounting assembly is detached from the mounting part, and the detection target module may be closer to the sensor module in the first state than in the second state.

In the other embodiment, the magnet carrier may include an elastic member configured to provide an elastic force to bring the first magnet proximal to the magnetic sensor when an external force greater than or equal to a predetermined level is not applied to the first magnet.

In the other embodiment, an end portion of the elastic member may be fixed and the first magnet may be coupled to another displaceable end portion of the elastic member.

In the other embodiment, the first magnet may be spaced apart from the magnetic sensor in the first state and positioned proximal to the magnetic sensor in the second state.

In the other embodiment, the magnet carrier may include an elastic member configured to provide an elastic force to cause the first magnet to be spaced apart from the magnetic sensor when an external force greater than or equal to a predetermined level is not applied to the first magnet.

In the other embodiment, the first magnet may be positioned proximal to the magnetic sensor in the first state and spaced apart from the magnetic sensor in the second state.

In the other embodiment, a motor pack, which is configured to provide a driving force to a surgical instrument, may include a mounting part configured to provide an area in which a mounting assembly, including a detection target module, is mounted, and a sensor module disposed adjacent to the mounting part, where the sensor module may include a magnetic sensor and a magnetic body having a first area adjacent to the magnetic sensor and a second area adjacent to the mounting part.

In the other embodiment, the motor pack may include a first state in which the mounting assembly is mounted on the mounting part and a second state in which the mounting assembly is detached from the mounting part, and the sensor module may be configured to detect the detection target module when the motor pack is in the first state.

In the other embodiment, the magnetic body may be magnetized while the magnet of the detection target module is in a state of being proximal to the magnetic body.

In the other embodiment, an adapter, mounted on a motor pack including a sensor module having a magnetic sensor and a magnetic body, may include a detection target module including a magnet disposed at a position corresponding to the magnetic body.

In the other embodiment, when the adapter is mounted on the motor pack and the detection target module is brought close to the sensor module, the magnet may magnetize the magnetic body.

Other aspects, features, and advantages of the disclosure will become better understood through the accompanying drawings, the appended claims, and the detailed description.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a conceptual diagram illustrating a surgical robot system including a surgical robot, according to an embodiment.

FIG. 2 is a perspective view of the surgical robot of FIG. 1.

FIG. 3 is an enlarged perspective view illustrating a partial configuration of a first arm of FIG. 2.

FIGS. 4 and 5 are diagrams schematically illustrating a mounting assembly detection system according to a first embodiment.

FIGS. 6 and 7 are perspective views illustrating a state in which a mounting assembly is detached from a motor pack, according to the first embodiment.

FIG. 8 is a side view of the motor pack and the mounting assembly of FIG. 7.

FIG. 9 is a side view illustrating a state in which the mounting assembly of FIG. 7 is mounted on the motor pack.

FIG. 10 is a side view illustrating a state in which a housing is removed from the motor pack of FIG. 9.

FIG. 11 is a side cross-sectional view illustrating a cross-section of the motor pack and the mounting assembly of FIG. 8.

FIG. 12 is a side cross-sectional view illustrating a cross-section of the motor pack and the mounting assembly of FIG. 9.

FIG. 13 is a rear view illustrating the motor pack of FIG. 10 when viewed from the rear.

FIGS. 14 and 15 are diagrams schematically illustrating a mounting assembly detection system according to a second embodiment.

FIG. 16 is a side cross-sectional view illustrating a cross-section of a motor pack and a mounting assembly of FIG. 14.

FIG. 17 is a side cross-sectional view illustrating a cross-section of a motor pack and a mounting assembly of FIG. 15.

FIGS. 18 and 19 are diagrams schematically illustrating a mounting assembly detection system according to a modification of the second embodiment.

### DETAILED DESCRIPTION

Hereinafter, the following embodiments will be described in detail with reference to the accompanying drawings. When describing the embodiments with reference to the accompanying drawings, the same or corresponding elements are denoted by the same reference numerals and redundant descriptions thereof are omitted.

Because various changes may be made to the present embodiments, specific embodiments will be illustrated in the drawings and described in detail in the detailed description. Effects and features of the present embodiments, and methods of achieving them will be clarified with reference to the detailed description below along with the drawings. However, the present embodiments are not limited to the embodiments disclosed below and may be implemented in various forms.

In describing the disclosure, when the detailed description of the relevant known technology is determined to obscure the gist of the disclosure, the detailed description thereof may be omitted.

The singular forms as used herein are intended to include the plural forms as well unless the context clearly indicates otherwise. While the terms such as "first" and "second" may be used to describe various elements, the elements should not be limited by the terms. These terms are only used to distinguish one element from another.

In the following embodiments, it will be understood that the terms "include" and/or "comprise" used herein specify the presence of stated features or elements, but do not preclude the presence or addition of one or more other features or elements.

In the following embodiments, it will be understood that, when a portion such as unit, region, or element is referred to as being "on" another portion, this may include not only a case where the portion is directly on the other portion, but also a case where intervening units, regions, or elements may be present therebetween.

In the following embodiments, it will be understood that the terms "connection" or "coupling" do not necessarily mean "direct and/or fixed connection or coupling" of two members, unless the context clearly indicates otherwise, and this does not preclude the disposition of other members between the two members.

Also, sizes of elements in the drawings may be exaggerated or reduced for convenience of explanation. For example, because sizes and thicknesses of elements in the drawings are arbitrarily illustrated for convenience of explanation, the following embodiments are not limited thereto.

The x-axis, the y-axis, and the z-axis are not limited to three axes of the rectangular coordinate system and may be interpreted in a broader sense. For example, the x-axis, the y-axis, and the z-axis may be perpendicular to one another or may represent different directions that are not perpendicular to one another.

When a certain embodiment is implemented differently, a specific process sequence may be performed differently from a sequence described herein. For example, two consecutively described processes may be performed substantially at the same time or performed in an order opposite to the stated order.

First, a surgical robot 10 capable of adopting a motor pack according to an embodiment is described.

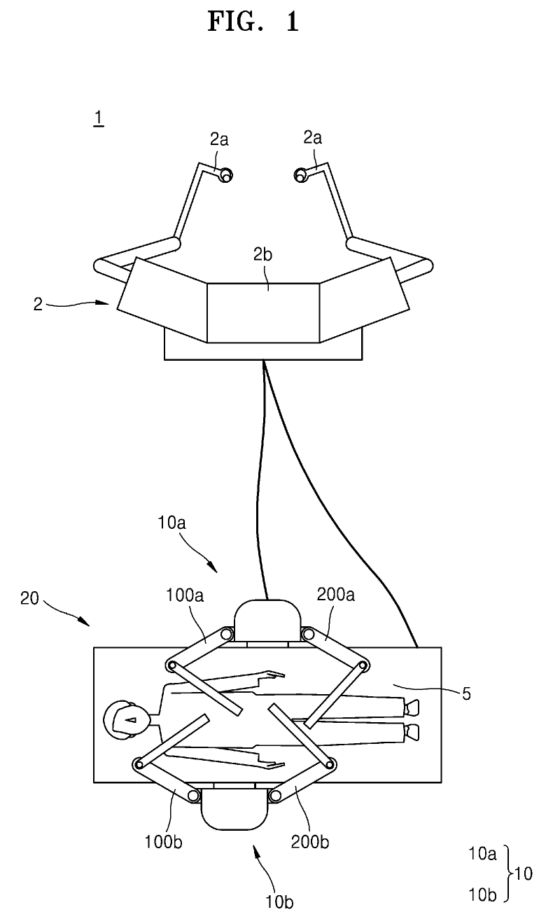

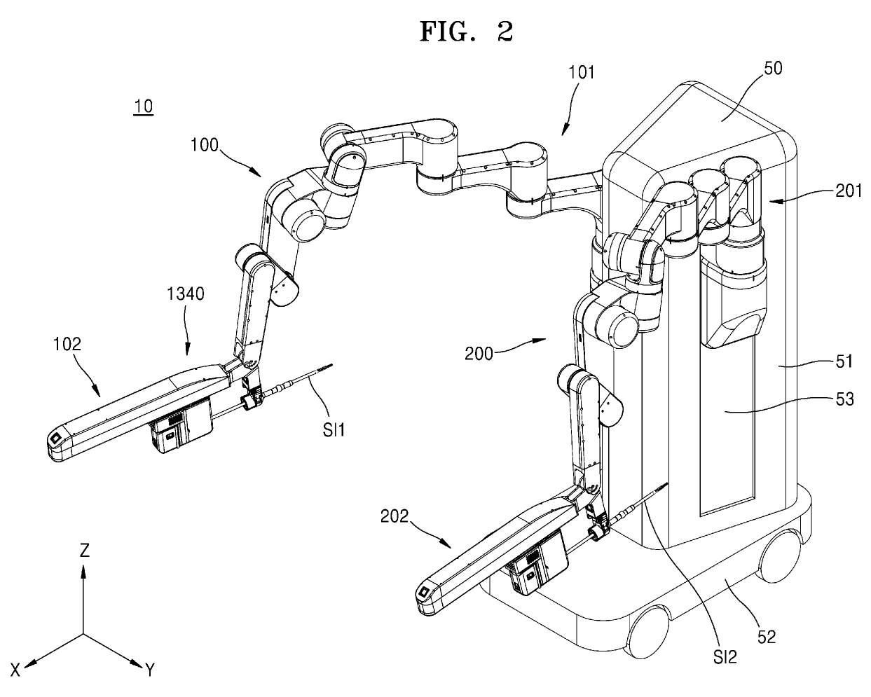

FIG. 1 is a conceptual diagram illustrating a surgical robot system including a surgical robot, according to an embodiment. FIG. 2 is a perspective view of the surgical robot 10 of FIG. 1.

Referring to FIG. 1 and FIG. 2, the surgical robot system 1 may include a master robot 2 and a surgical robot 10.

The master robot 2 may include a manipulation member and a display member, and the surgical robot 10 may include one or more robot arms 100 and 200.

The master robot 2 may include manipulation members 2a which allows an operating surgeon to hold and manipulate the master robot 2 with both hands. A display member 2b of the master robot 2 may display an image captured using a laparoscope as a video image. In some embodiments, a virtual manipulation panel may be displayed on the display member 2b together with the image captured using the laparoscope or the like, or may be displayed independently. A detailed description of the layout, configuration, or the like of the virtual manipulation panel is omitted.

In some embodiments, the surgical robot 10 may include at least two robot arms 100 and 200. The robot arms 100 and 200 may be provided in a module form which is operable independently of each other, and an algorithm for preventing collision between the robot arm units 100 and 200 may be applied to the surgical robot system 1.

The surgical robot system 1 may include one or more surgical robots 10. FIG. 1 illustrates an embodiment in which the surgical robot system 1 includes two surgical robots 10a and 10b each having two robot arms 100 and 200, that is, a total of four robot arms 100a, 200a, 100b, and 200b are disposed.

In an embodiment, surgical instruments may be attached to two or more of the robot arms 100 and 200, and laparoscopes may be attached to one or more of the robot arms 100 and 200. An operating surgeon may select the robot arms 100a, 200a, 100b, and 200b to be controlled through the master robot 2. By allowing the operating surgeon to directly control three or more surgical instruments using the master robot 2, the operating surgeon may accurately and freely control multiple instruments on a surgical bed 5 according to the intention of the operating surgeon without the need for a surgical assistant.

A detailed configuration and operating principle of the surgical robot 10 are described in detail.

Referring to FIG. 2, the surgical robot 10 may include a main body part 50, a first arm 100, and a second arm 200. FIG. 2 illustrates an embodiment in which the surgical robot 10 includes two robot arms, and the respective robot arms are defined as the first arm 100 and the second arm 200.

The main body part 50 may act as the body of the surgical robot 10, and the first arm 100 and the second arm 200 may be disposed in the main body part 50. In some embodiments, the main body part 50 may provide reference points for driving the first arm 100 and the second arm 200.

The main body part 50 may include a first main body 51 and a second main body 52. The first arm 100 and the second arm 200 may be disposed in the first main body 51. The second main body 52 may support the first main body 51. In some embodiments, the second main body 52 may have wheels, as illustrated in FIG. 2. Due to this, the surgical robot 10 may be movable.

The main body part 50 may have an elevating guide 53. The elevating guide 53 may be provided to correspond to the number of robot arms disposed in the main body part 50. The elevating guide 53 may be formed concavely on a side of the main body part 50, and the respective robot arms 100 and 200 may be coupled to the elevating guide 53 and slidingly movable in a first direction.

In describing the disclosure, a portion close to the main body part 50 is referred to as a proximal end, and a portion far from the main body part 50 is referred to as a distal end. For example, a portion of the first arm 100 which is close to the main body part 50 is defined as a proximal end 101 of the first arm 100, and a portion of the first arm 100 which is far from the main body part 50 is defined as a distal end 102 of the first arm 100. Similarly, a portion of the second arm 200 which is close to the main body part 50 is defined as a proximal end 201 of the second arm 200, and a portion of the second arm 200 which is far from the main body part 50 is defined as a distal end 202 of the second arm 200.

The first arm unit 100 may be disposed on a side of the main body part 50, and a first surgical instrument SI1 may be mounted on the first arm 100. The surgical robot 10 may drive the first arm 100 to control the position and posture of the first surgical instrument SI1.

A first arm connection part (not shown) may have a plurality of connection links, and the posture of the first arm connection part (not shown) may be determined according to the driving of the respective connection links. A remote center of motion (RCM) point of the first surgical instrument SI1 may be determined according to the posture of the first arm connection part (not shown). In this case, the RCM point of the first surgical instrument SI1 may refer to a virtual center point which serves as a reference for the rotation of the first surgical instrument SI1. The first surgical instrument SI1 may be rotated around the RCM point to perform a yaw motion and a pitch motion.

The second arm 200 may be disposed on another side of the main body part 50, and a second surgical instrument SI2 may be mounted on the second arm 200. The surgical robot 10 may drive the second arm 200 to control the position and posture of the second surgical instrument SI2.

A second arm connection part (not shown) may have a plurality of connection links, and the posture of the second arm connection part (not shown) may be determined according to the driving of the respective connection links. An RCM point of the second surgical instrument SI2 may be determined according to the posture of the second arm connection part (not shown). In this case, the RCM point of the second surgical instrument SI2 may refer to a virtual center point which serves as a reference for the rotation of the second surgical instrument SI2. The second surgical instrument SI2 may be rotated around the RCM point to perform a yaw motion and a pitch motion.

The robot arms may each include a plurality of arm connection links and a plurality of arm extension links. The arm connection link and the arm extension link may be respectively rotated around reference axes thereof. Due to the rotation motion, the robot arms may control the posture and layout of the surgical instrument within a movable range.

A detailed description of the arm connection links and the arm extension links is omitted herein.

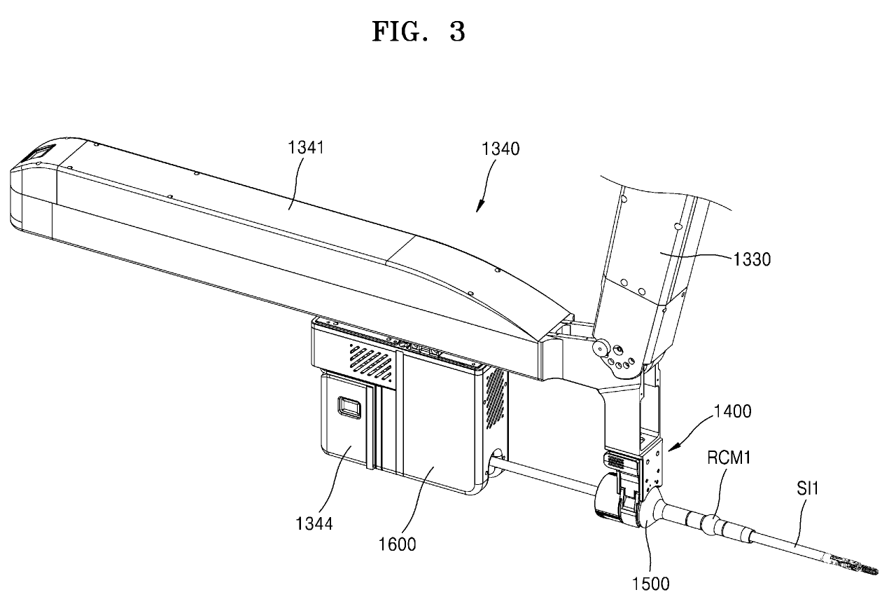

FIG. 3 is an enlarged perspective view illustrating a partial configuration of the first arm 100 of FIG. 2.

Referring to FIGS. 2 and 3, the first arm 100 according to an embodiment may include a first arm slide link 1340 on the distal end 102 side.

The first arm slide link 1340 may slidingly move the first surgical instrument SI1.

The first arm slide link 1340 may be coupled to another end of a first-arm third-extension link 1330, that is, on a distal end side, and the first surgical instrument SI1 may be disposed in the first arm slide link 1340.

The first arm slide link 1340 may include a translation arm 1341, a slide motor pack 1600, a slide driving unit 1344, and a trocar holder 1400.

The translation arm 1341 may be coupled to another end of the first-arm third-extension link 1330 and may be moved together with the first-arm third-extension link 1330. For example, when the first-arm third-extension link 1330 is driven, the posture of the translation arm 1341 may be changed together.

The slide motor pack 1600 may provide a driving force for the slide motion of the first surgical instrument SI1. The slide motor pack 1600 may include one or more first motors and other components capable of producing and transmitting power.

The slide driving unit 1344 may receive the driving force from the slide motor pack 1600 and slidingly move the first surgical instrument SI1. The first surgical instrument SI1 may have an end connected to the slide driving unit 1344 and may be linearly moved by the slide driving unit 1344.

The trocar holder 1400 may be disposed in an area of the translation arm 1341, and a trocar 1500 may be mounted on the trocar holder 1400. For example, the trocar holder 1400 may be disposed close to an end portion of the first-arm third-connection link 1330 on the distal end side.

The trocar 1500 may be mounted on the trocar holder 1400, and the first surgical instrument SI1 may be coupled to the trocar 1500. The first surgical instrument SI1 may be mounted by passing through the trocar 1500. In some embodiments, the first surgical instrument SI1 may be partially supported on the trocar 1500 and may be slidingly moved.

An RCM point RCM1 of the first surgical instrument SI1 may be formed on a side of the trocar 1500. For example, the trocar 1500 may provide an RCM point, which is a reference point for rotation, including a yaw motion and a pitch motion of the first surgical instrument SI1, on a side. When the posture of the first arm connection part is determined, the position of the RCM point disposed on the trocar 1500 may also be determined, and even when the first surgical instrument SI1 is slidingly moved, the position of the RCM point may be fixed.

Hereinafter, a motor pack and a mounting assembly, according to an embodiment, is described in detail.

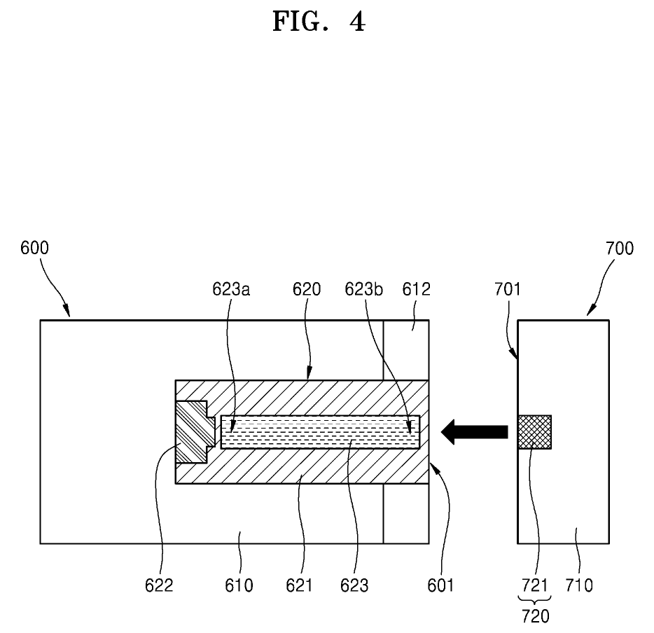

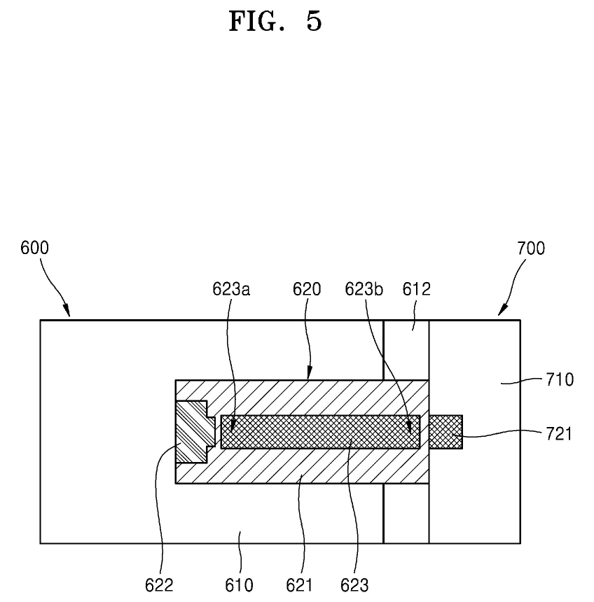

FIGS. 4 and 5 are diagrams schematically illustrating a mounting assembly detection system according to a first embodiment. FIG. 4 is a diagram illustrating a state before the mounting assembly is mounted on the motor pack, and FIG. 5 is a diagram illustrating a state after the mounting assembly is mounted on the motor pack.

Referring to FIGS. 4 and 5, the mounting assembly detection system according to the first embodiment may include a motor pack 600 and a mounting assembly 700.

An example in which the motor pack 600 is adopted in the surgical robot 10 described above is described. However, the concept of the disclosure is not limited thereto, and the motor pack 600 may be a motor pack 600 adopted in a handheld surgical instrument product.

The motor pack 600 may provide a driving force for controlling the surgical instrument. For example, a driving unit of the surgical instrument may receive the driving force from the motor pack 600 and operate a pulley and a wire.

The motor pack 600 may include one or more driving motors (not shown) and other components capable of producing and transmitting power.

The mounting assembly 700 may be mounted on the motor pack 600. The mounting assembly 700 may include, for example, an adapter for a surgical instrument and/or a drape. In an embodiment, after the adapter is mounted on the motor pack 600, the surgical instrument may be mounted on the adapter.

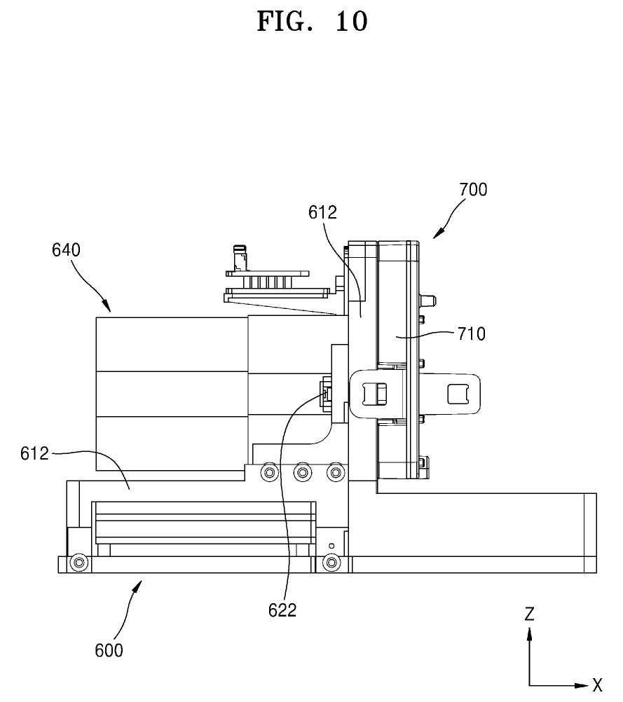

The motor pack 600 may include a driving motor 640 (see FIG. 10), a mounting part 612, and a sensor module 620, which are provided in a main body.

The driving motor 640 may be mounted on the mounting part 612, and the mounting part 612 may provide an area in which the mounting assembly 700 is mounted. In other words, the mounting part 612 may include a first surface 601 which comes into contact with the mounting assembly 700.

As illustrated in FIGS. 4 and 5. the sensor module 620 may be disposed adjacent to the mounting part 612. From another viewpoint, at least one area of the sensor module 620 may be supported and fixed to the mounting part 612. For example, the sensor module 620 may be mounted on the mounting part 612.

The sensor module 620 is a device which detects whether the mounting assembly 700 is mounted on the motor pack 600. Hereinafter, the sensor module 620 and a detection target module 720 of the mounting assembly 700 are described in detail.

The sensor module 620 according to the first embodiment may include a magnetic sensor 622, a magnetic body 623, and a sensor housing 621.

The sensor housing 621 may form the outer shape of the sensor module 620 and may accommodate the magnetic sensor 622 and the magnetic body 623 to be described below. In some embodiments, at least a portion of the sensor housing 621 may be coupled to the mounting part 612 so that the sensor module 620 may be fixed to a motor pack body 610.

The magnetic sensor 622 may be spaced apart from the mounting part 612 by a preset distance. From another viewpoint, when the mounting assembly 700 is mounted on the motor pack 600, the magnetic sensor 622 may be spaced apart from a magnet 721 of the detection target module 720 to be described below by a preset distance. The magnetic body 623 may be disposed between the magnetic sensor 622 and the first surface 601 of the mounting part 612. In other words, the magnetic body 623 may be disposed closer to the mounting assembly 700 than the magnetic sensor 622.

In an embodiment, the magnetic sensor 622 may be a Hall effect sensor. The Hall effect sensor is a device which detects a change in magnetic field and converts the detected change into an electrical signal. The Hall effect sensor may detect the position or speed of the magnet in a non-contact manner. Accordingly, the Hall effect sensor may detect a target object, including the magnet, through the change in magnetic field caused by the movement of the magnet.

According to the related art, a separate pin protrudes outward to move a magnet toward a magnetic sensor. When the protruding pin is pressed by an external force and the magnet provided at an end of the pin is moved toward the magnetic sensor, the magnetic sensor detects a change in magnetic field. However, when the method of pressing the protruding pin is used, there is a concern that the pin may be damaged during the process in which the mounting assembly (e.g., the adapter) presses the protruding pin, and when the pin is pressed incorrectly, there may occur a recognition error in which the adapter is recognized as being mounted.

Embodiments of the disclosure provide a method of sensing the mounting assembly 700 without externally exposed detection pins or the like.

The mounting assembly 700 according to the first embodiment may include the detection target module 720 which is an object to be detected by the magnetic sensor 622.

The detection target module 720 may include the magnet 721 disposed at a position corresponding to the sensor module 620. The magnet 721 may be disposed opposite to the magnetic body 623 provided in the sensor module 620. For example, when the mounting assembly 700 is mounted on the mounting part 612, the magnet 721 of the detection target module 720 may be disposed at a position corresponding to a second area 623b of the magnetic body 623.

From another viewpoint, when a state in which the mounting assembly 700 is mounted on the mounting part 612 is defined as a first state and a state in which the mounting assembly 700 is detached from the mounting part 612 is defined as a second state, the detection target module 720 may be closer to the sensor module 620 in the first state than in the second state.

When the mounting assembly 700 is in the first state, the magnet 721 of the detection target module 720 may be closest to the magnetic body 623.

A first area 623a of the magnetic body 623 may be disposed adjacent to the magnetic sensor 622, and the second area 623b may be disposed adjacent to the mounting part 612. From another viewpoint, the magnetic body 623 may be disposed between the mounting part 612 and the magnetic sensor 622 spaced apart from the mounting part 612.

For example, the magnetic body 623 may be a longitudinal member extending from the first area 623a to the second area 623b. The second area 623b of the magnetic body 623 may be a portion which comes into contact with the magnet 721 of the detection target module 720.

The magnetic body 623 provided in the sensor module 620 according to the first embodiment may be a magnetic body 623 in a non-magnetized state. As illustrated in FIG. 4, in a basic state in which the mounting assembly 700 is not mounted on the motor pack 600, the magnetic body 623 may be in a non-magnetized state.

For example, as illustrated in FIG. 5, the magnetic body 623 may be magnetized in a state in which the magnet 721 is close to the magnetic body 623 of the sensor module 620.

In other words, the magnetic body 623 becomes temporarily magnetized under the influence of the magnet 721, and as a result, the magnetism of the magnet 721 may extend to the magnetic sensor 622.

Accordingly, because the magnetic body 623 becomes magnetized, the magnetic sensor 622 may detect a change in magnetic field, and as a result, the sensor module 620 may detect the detection target module 720.

As such, because the magnetic body 623 becomes magnetized, the magnetic sensor 622 may detect the detection target module 720.

Accordingly, even when the sensor module 620 according to the first embodiment does not include the magnet that involves a change in physical position, the magnetic sensor 622 may detect a change in magnetic field while the detection target module 720 is adjacent to the sensor module 620.

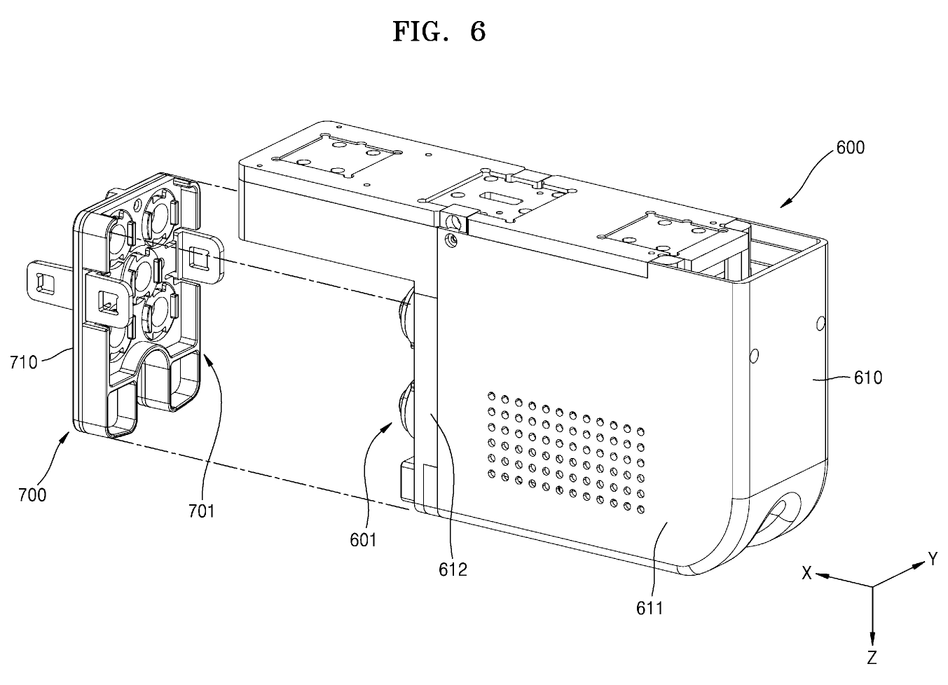

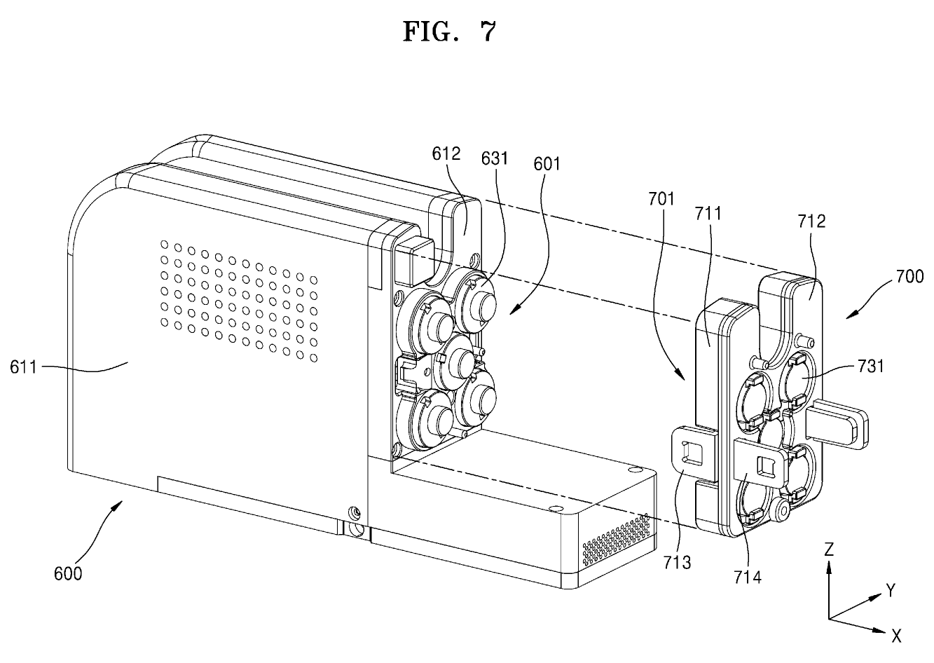

Hereinafter, the motor pack 600 and the mounting assembly 700 according to the first embodiment are described in detail below with reference to FIGS. 6 and 7.

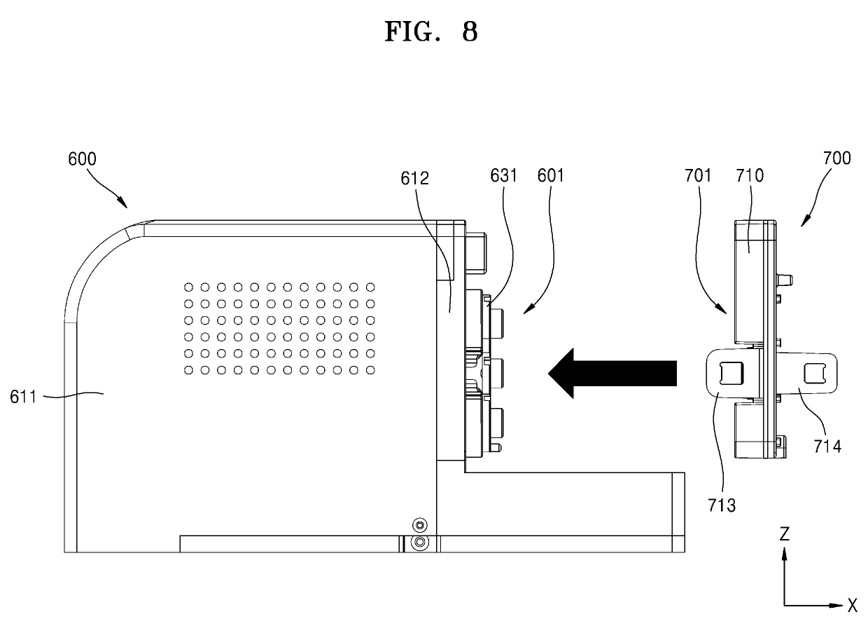

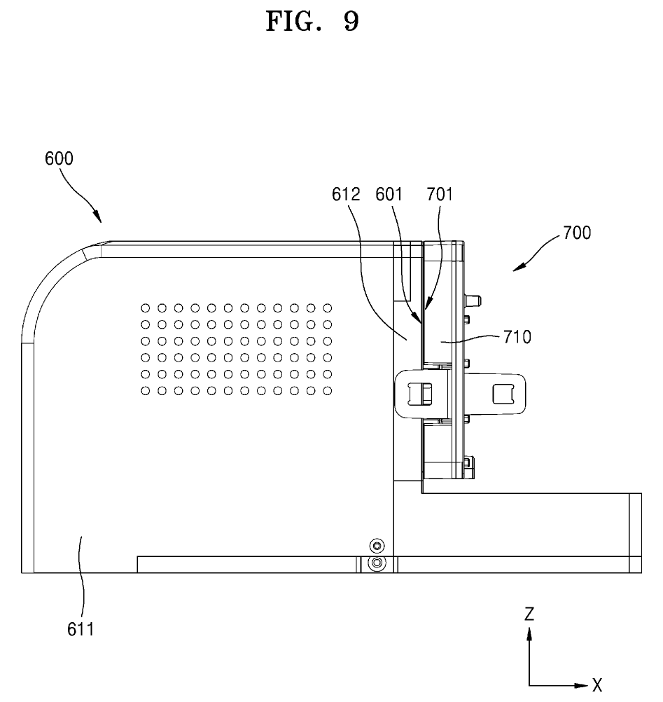

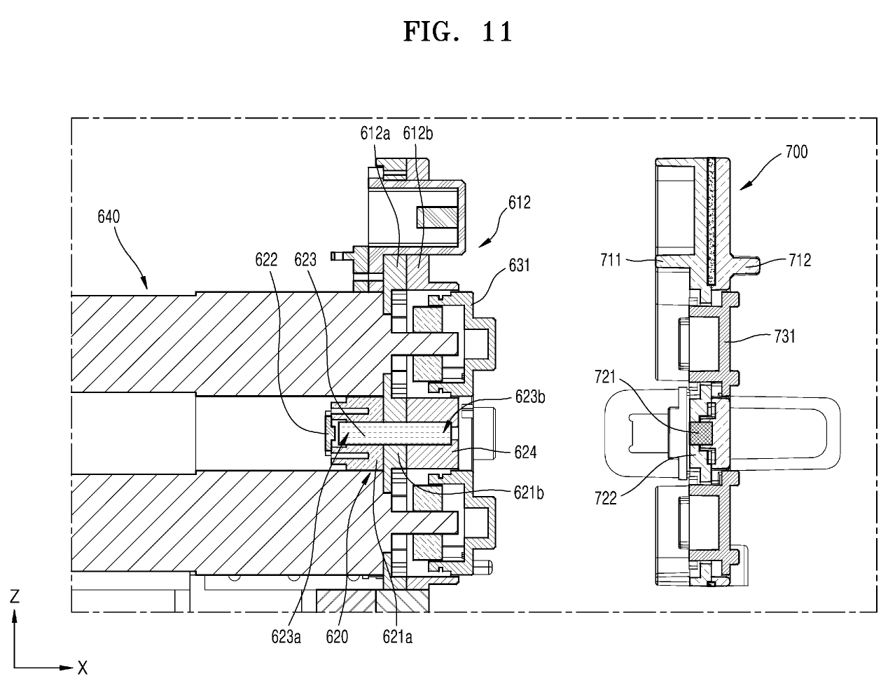

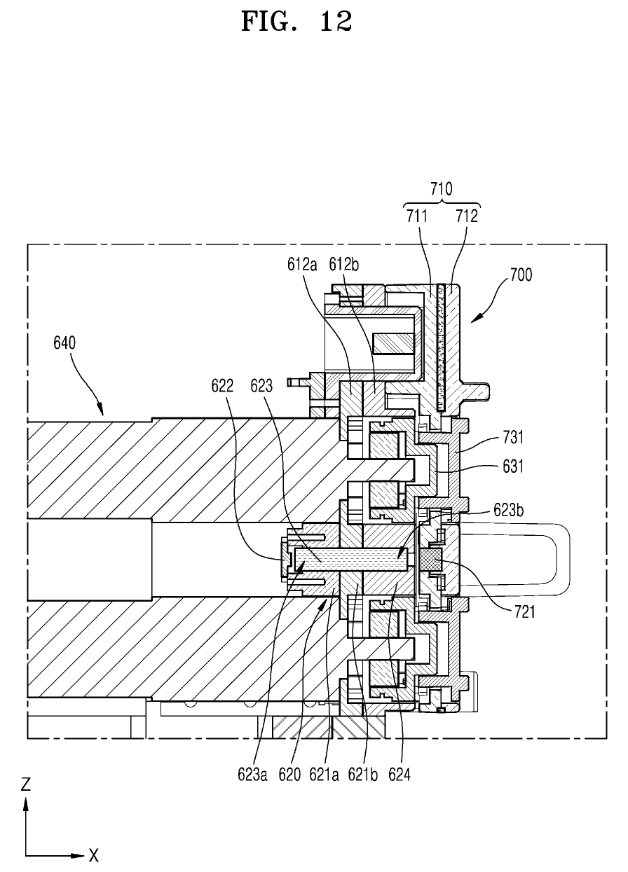

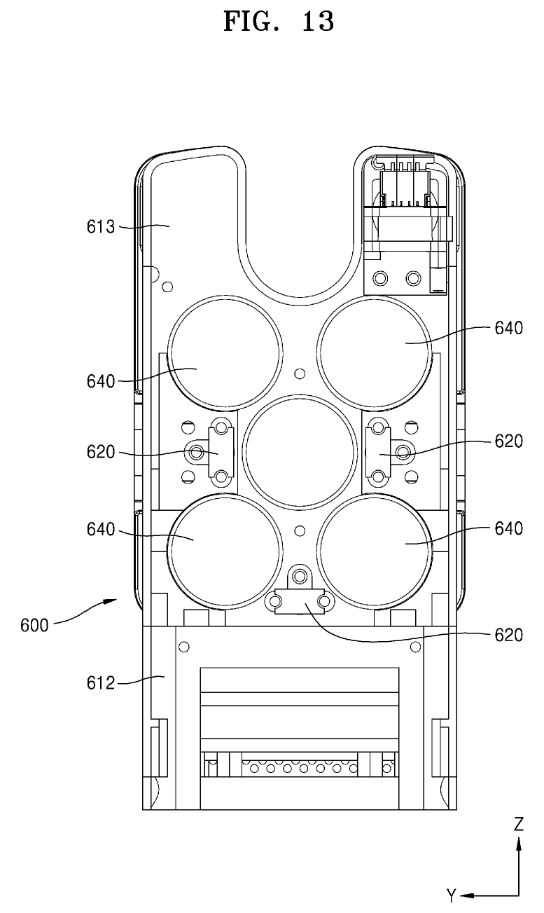

FIG. 6 is a perspective view illustrating the motor pack 600 and the mounting assembly 700 according to the first embodiment, and FIG. 7 is a perspective view illustrating the motor pack 600 and the mounting assembly 700 of FIG. 6 when viewed from a different angle. FIGS. 6 and 7 illustrate a state in which the mounting assembly 700 is detached from the motor pack 600. FIG. 8 is a side view of the motor pack 600 and the mounting assembly 700 of FIG. 7, and FIG. 9 is a side view illustrating a state in which the mounting assembly 700 of FIG. 7 is mounted on the motor pack 600. FIG. 10 is a side view illustrating a state in which a housing is removed from the motor pack 600 of FIG. 9. FIG. 11 is a side cross-sectional view illustrating a cross-section of the motor pack 600 and the mounting assembly 700 of FIG. 8, and FIG. 12 is a side cross-sectional view illustrating a cross-section of the motor pack 600 and the mounting assembly 700 of FIG. 9. FIG. 13 is a rear view illustrating the motor pack 600 of FIG. 10 when viewed from the rear.

Referring to FIGS. 6 to 13, the motor pack 600 according to the first embodiment may further include a main body housing 611, a base frame 614, a driving motor 640, a mounting part frame 612a, a mounting part cover 612b, a pulley head part 631, and the like.

The base frame 614 may constitute the frame of the motor pack body 610 and may be coupled to the mounting part 612. Various electronic devices and components related to the operation of the driving motor 640 may be accommodated or disposed in the base frame 614.

The driving motor 640 may be provided in plurality, and the plurality of driving motors 640 may be disposed parallel to each other. The driving motor 640 may be disposed parallel to the longitudinal direction in which the mounting assembly 700 is mounted. For example, the driving motor 640 may be coupled to the mounting part frame 612a of the mounting part 612.

The driving motor 640 may be mounted to be disposed parallel to a direction extending from a surface opposite to the first surface 601 of the mounting part 612 facing the mounting assembly 700.

The main body housing 611 may be fastened to the base frame 614 and the mounting part 612 of the motor pack 600 to form the outer shape of the motor pack 600. The main body housing 611 may protect internal components of the motor pack 600, including the driving motor 640.

The mounting part 612 may include the mounting part frame 612a and the mounting part cover 612b. The mounting part frame 612a may be a portion on which the driving motor 640 described above is mounted.

For example, the mounting part frame 612a may have a through hole through which an end of the driving motor 640 passes. For example, a drive pulley which receives power from the driving motor 640 and performs a rotational motion may be inserted through the through hole. The driving pulley may be coupled to a pulley head part 631 which surrounds the outer surface of the driving pulley.

The pulley head part 631 may be coupled to a driving input part of the mounting assembly 700 and may transmit a driving force of the driving motor 640. To this end, the pulley head part 631 may form a protrusion or a coupling groove.

The mounting part cover 612b may be disposed to correspond to the mounting part frame 612a and may cover a surface of the mounting part frame 612a. For example, the mounting part cover 612b may have a through hole corresponding to the shape of the pulley head part 631 and accommodating at least a portion of the pulley head part 631. The mounting part cover 612b may provide a surface to which the mounting assembly 700 is coupled. In other words, the mounting assembly 700 may be coupled to a first surface 601 of the mounting part cover 612b.

For example, the sensor module 620 may be mounted on the mounting part 612 as described above. For example, the sensor module 620 may be disposed on a side surface of the mounting part frame 612a in which the driving motor 640 is disposed. Referring to FIG. 10, the sensor module 620 may be disposed between the driving motor 640 and the driving motor 640.

When a plurality of sensor modules 620 are provided, the sensor modules 620 may be spaced apart from each other by a preset distance. For example, the sensor modules 620 may be disposed symmetrically with one driving motor 640 therebetween. However, a method in which the sensor modules 620 are disposed is not limited thereto.

When the motor pack 600 includes the plurality of sensor modules 620, the mounting assembly 700 may include a plurality of detection target modules 720 corresponding to the plurality of sensor modules 620. The mounting assembly detection system may detect the mounting assembly 700 when the plurality of sensor modules 620 detect all the plurality of detection target modules 720. In other words, only when all the sensor modules 620 detect the detection target modules 720, the mounting assembly detection system may determine that the mounting assembly 700 is properly mounted on the mounting part 612.

The sensor housing 621 of the sensor module 620 may surround the magnetic sensor 622 and the magnetic body 623 and may accommodate the magnetic sensor 622 and the magnetic body 623. The sensor housing 621 may be formed to correspond to the longitudinal direction of the magnetic body 623. In an embodiment, the sensor housing 621 may be provided as a single member, or may be provided as different members coupled to each other. For example, a portion in which the magnetic sensor 622 is accommodated and a portion in which the magnetic body 623 is accommodated may be provided as different materials. In some embodiments, an area of the mounting part frame 612a and the mounting part cover 612b may be provided as the sensor housing 621. In other words, at least a portion of the magnetic body 623 may be inserted into the mounting part frame 612a and/or the mounting part cover 612b.

The sensor housing 621 may include a partition wall 624 disposed at an end portion of the magnetic body 623. From another viewpoint, the end portion of the magnetic body 623 may be in contact with the partition wall 624.

Therefore, a the magnetic body 623 may be spaced apart from the magnet 721 by a preset distance while the mounting assembly 700 is in a state of being mounted on the motor pack 600.

In other words, the partition wall 624 may be disposed between the magnet 721 and the magnetic body 623 while the mounting assembly 700 is in a state of being mounted on the mounting part 612. Therefore, the magnetic body 623 and the magnet 721 may be restricted from coming into direct contact with each other.

Referring again to FIGS. 8 and 9, as described above, the first area 623a of the magnetic body 623 may be adjacent to the magnetic sensor 622 and the second area 623b of the magnetic body 623 may be adjacent to the mounting part 612.

For example, the magnetic body 623 may be a longitudinal member parallel to the longitudinal direction in which the mounting assembly 700 is mounted. The magnetic body 623 may be formed to extend from the partition wall 624 of the sensor housing 621 formed in the mounting part cover 612b to the magnetic sensor 622 disposed on the side surface of the driving motor 640. In other words, the magnetic body 623 may be at least partially accommodated in the mounting part frame 612a and the mounting part cover 612b.

The mounting assembly 700 is an external device which may be mounted on the motor pack 600, and the mounting assembly 700 may include an adapter, a surgical instrument, and the like.

In describing the present embodiment, an example in which the mounting assembly 700 is an adapter is described. Referring again to FIGS. 6 to 12, the adapter may include a base body 711, an adapter cover 712, a detection target module 720, a pulley connection part 731, and fastening parts 713 and 714.

The base body 711 may be a portion which forms the outer shape of the adapter and to which the adapter cover 712 is coupled. A pulley accommodation part (not shown) in which the pulley connection part 731 is disposed may be formed in the base body 711. The pulley connection part 731 may be rotatable in a state of being inserted into the pulley accommodation part. For example, the pulley connection part 731 may be rotated around the same shaft as the rotation shaft of the driving motor 640.

For example, the pulley connection part 731 may be fastened to the pulley head part 631 of the motor pack 600 described above and may be rotated together with the pulley head part 631. Accordingly, the pulley connection part 731 may form a protrusion or a fastening groove which may be fastened to the pulley head part 631.

As illustrated, the adapter may include a plurality of pulley connection part 731 which are each disposed to correspond to the pulley head part 631 of the motor pack 600.

The base body 711 may include a second surface 701 opposite the first surface 601 of the mounting part 612 of the motor pack 600. In other words, the second surface 701 of the base body 711 may be an area facing the mounting part 612.

Similar to the base body 711, the adapter cover 712 may form a portion of the accommodation part of the pulley connection part 731. The adapter cover 712 may be coupled to the surface opposite the second surface 701 of the base body 711 and may provide a coupling surface to which the surgical instrument is coupled.

The fastening parts 713 and 714 may include a first fastening part 713 fastened to the motor pack 600 and a second fastening part 714 fastened to the surgical instrument. The first fastening part 713 and the second fastening part 714 may be formed to extend in opposite directions with respect to the coupling surface of the adapter cover 712. The first fastening part 713 and the second fastening part 714 may be respectively formed on opposite surfaces of the adapter. However, the concept of the disclosure is not limited thereto, and the position, shape, and number of the fastening parts of the adapter may vary.

For example, the detection target module 720 may be provided in an area of the base body 711 of the adapter. For example, the detection target module 720 may be disposed on the base body 711 to be opposite the sensor module 620 of the motor pack 600.

The detection target module 720 may include a magnet housing 722 and a magnet 721. The magnet housing 722 may be a portion which surrounds the magnet 721 and accommodates the magnet 721.

The magnet housing 722 may be mounted on the base body 711 in a state of being accommodating the magnet 721, or the magnet housing 722 may be formed on the base body 711. In other words, the magnet housing 722 may be provided as a separate member, or may be a space formed in the base body 711 itself.

The magnet housing 722 may be formed on the base body 711 and the adapter cover 712 of the adapter, but the disclosure is not necessarily limited thereto.

As described above, the magnet 721 may be disposed at a position corresponding to the magnetic body 623 of the sensor module 620. The magnet 721 may be provided as an area protruding from the base body 711, or may be provided to be built into the base body 711 so as not to protrude to the outside.

In another embodiment, the motor pack 600 may further include, in addition to the sensor module 620 which detects whether the adapter is mounted, a sensor module which detects whether the surgical instrument is mounted.

For example, when the sensor module which detects whether the adapter is mounted is defined as a first sensor module and the sensor module which detects whether the surgical instrument is mounted is defined as a second sensor module, the second sensor module may be mounted on the mounting part 612, like the first sensor module. The second sensor module may include substantially the same configuration as the first sensor module.

In other words, the second sensor module may include the magnetic sensor 622 and the magnetic body 623. However, for convenience of explanation, the magnetic sensor 622 provided in the second sensor module is referred to as the second magnetic sensor, and the magnetic body 623 provided in the second sensor module is referred to as the second magnetic body.

The mounting assembly according to the present embodiment may be an adapter disposed between the surgical instrument and the motor pack 600. For example, the adapter may be mounted on the motor pack 600, and the surgical instrument may be mounted on the adapter.

The mounting assembly (adapter) according to the present embodiment may include a bridge module. The bridge module may have a third magnetic body disposed at a position corresponding to the second sensor module.

The surgical instrument may include a second magnet at a position corresponding to the bridge module.

When the adapter is mounted on the motor pack 600, the bridge module of the adapter may be brought close to the second sensor module of the motor pack 600, and thus, the third magnetic body of the bridge module may be brought close to the second magnetic body of the sensor module. In other embodiments, the third magnetic body and the second magnetic body may come into direct contact with each other.

When the surgical instrument is also mounted on the adapter, the second magnet of the surgical instrument may be brought close to the third magnetic body of the bridge module. Therefore, the third magnetic body becomes temporarily magnetized under the influence of the second magnet, and as a result, the magnetism of the second magnet may extend to the third magnetic body.

The second magnetic body also becomes temporarily magnetized under the influence of the third magnetic body, and as a result, the magnetism of the second magnet may extend to the second magnetic body.

In other words, while the adapter and the surgical instrument are in a state of being mounted on the motor pack 600, the third magnetic body and the second magnetic body may be magnetized by the second magnet provided in the surgical instrument.

Because the second magnetic body becomes magnetized, the second magnetic sensor may detect a change in magnetic field, and as a result, the second sensor module may detect the bridge module. As described above, because the second magnetic body is magnetized, the second magnetic sensor may detect the bridge module.

Hereinafter, a mounting assembly detection system according to a second embodiment is described.

In the mounting assembly detection system according to the second embodiment, a configuration of a sensor module 1620 is characteristically different, compared to the mounting assembly detection system according to the first embodiment described with reference to FIG. 4 and the like. Hereinafter, the configuration which is changed, compared to the mounting assembly detection system according to the first embodiment, is described in detail.

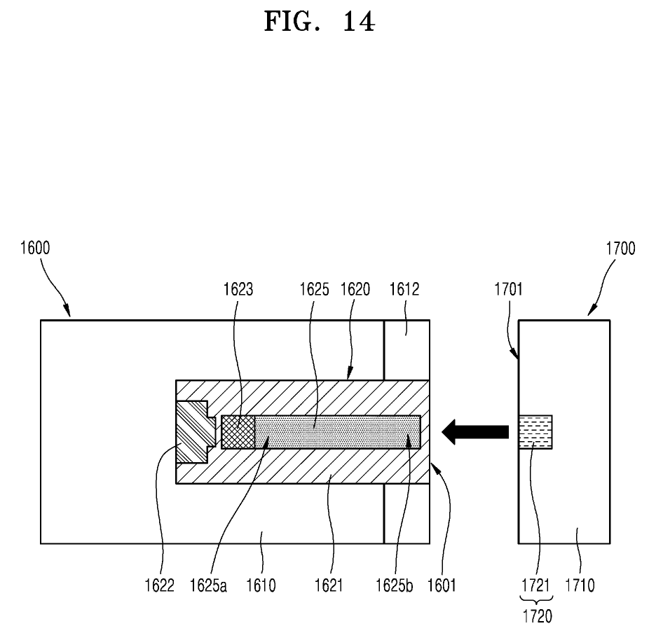

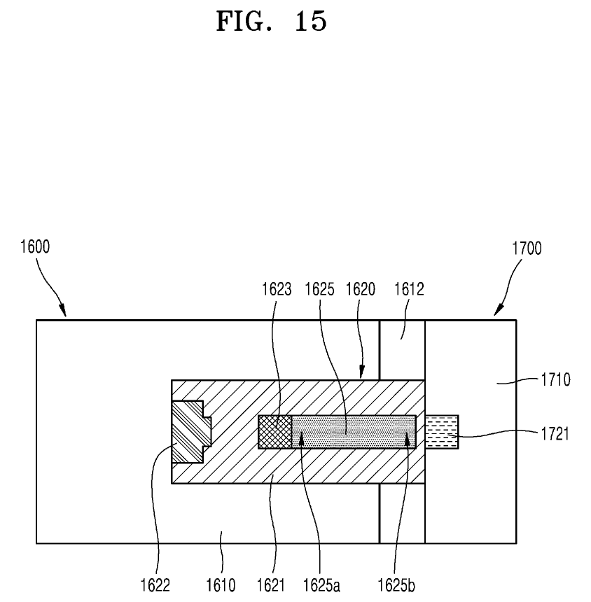

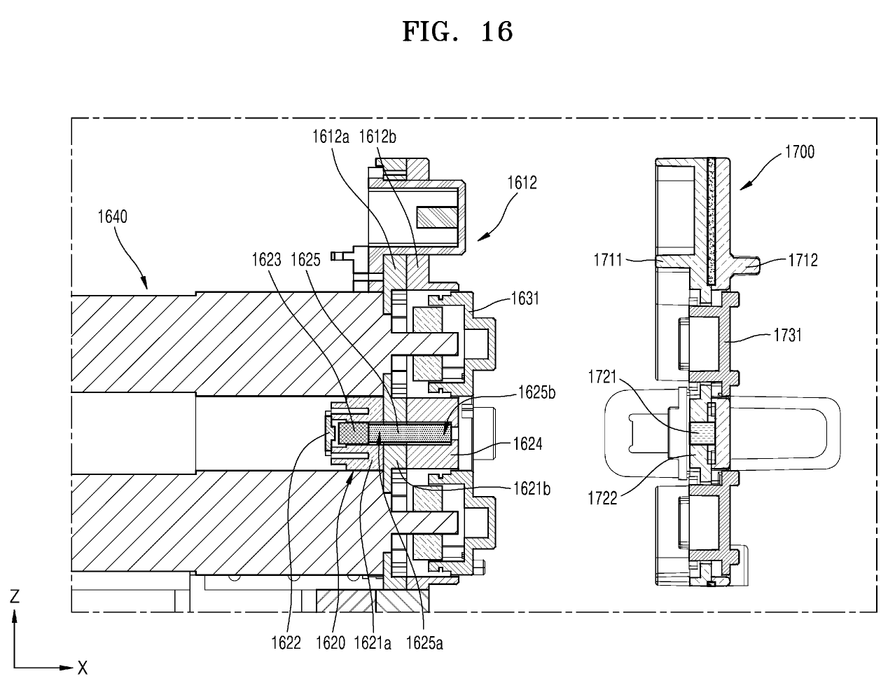

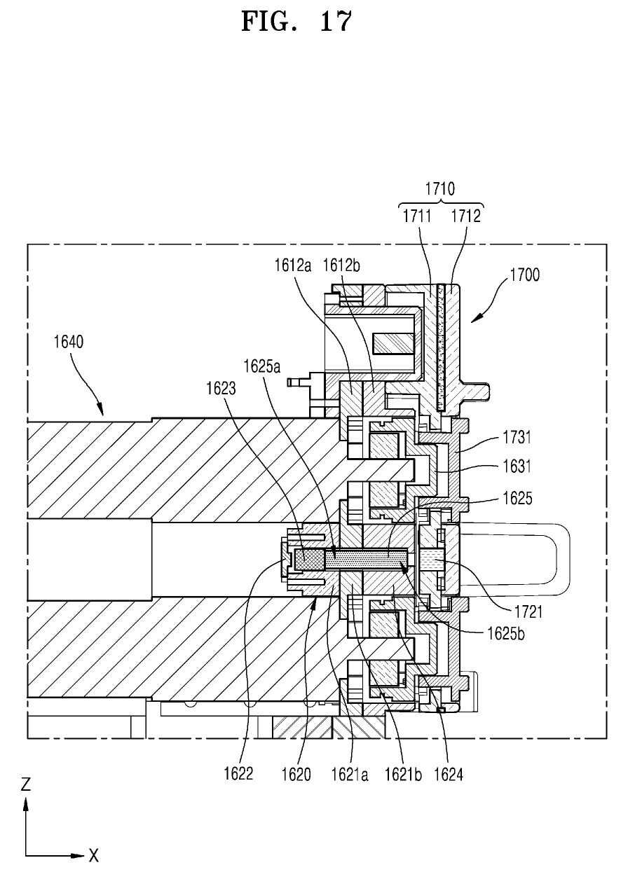

FIGS. 14 and 15 are diagrams schematically illustrating the mounting assembly detection system according to the second embodiment. Specifically, FIG. 14 is a diagram illustrating a state before a mounting assembly 1700 is mounted on a motor pack 1600, and FIG. 15 is a diagram illustrating a state after the mounting assembly 1700 is mounted on the motor pack 1600. FIG. 16 is a side cross-sectional view illustrating a cross-section of the motor pack 1600 and the mounting assembly 1700 of FIG. 14, and FIG. 17 is a side cross-sectional view illustrating a cross-section of the motor pack 1600 and the mounting assembly 1700 of FIG. 15.

Referring to FIGS. 9 and 10 and FIGS. 16 and 17, the motor pack 1600 according to the second embodiment may further include a main body housing 611 (see FIG. 9), a base frame 614 (see FIG. 10), a driving motor 1640, a mounting part frame 1612a, a mounting part cover 1612b, and a pulley head part 1631. The adapter may include a base body 1711, an adapter cover 1712, a detection target module 1720, a pulley connection part 1731, and a fastening part.

The main body housing, the base frame, the driving motor 1640, the mounting part frame 1612a, the mounting part cover 1612b, the pulley head part 1631, the base body 1711, the adapter cover 1712, the detection target module 1720, the pulley connection part 1731, and fastening part according to the present embodiment are substantially the same as the main body housing 611, the base frame 614, the driving motor 640, the mounting part frame 612a, the mounting part cover 612b, the pulley head part 631, the base body 711, the adapter cover 712, the detection target module 720, the pulley connection part 731, and the fastening parts 713 and 714 according to the first embodiment, and therefore, a detailed description thereof is omitted herein.

The sensor module 1620 may further include, in addition to a magnetic sensor 1622 and a sensor housing 1621, a magnet 1623 and a magnet carrier 1625.

The magnet 1623 may be accommodated in the sensor housing 1621 and may be movable between a position adjacent to the magnetic sensor 1622 and a position spaced apart from the magnetic sensor 1622. For example, the magnet 1623 may be movable between a position adjacent to the magnetic sensor 1622 and a position spaced apart from the magnetic sensor 1622 while being coupled to the magnet carrier 1625 to be described below.

Accordingly, the magnetic sensor 1622 may detect a change in magnetic field according to the movement of the magnet 1623, and the sensor module 1620 may determine whether the mounting assembly 1700 is mounted, based on the change in magnetic field.

According to the embodiment illustrated in FIGS. 14 and 15, the magnet 1623 may be disposed proximal to the magnetic sensor 1622 in a basic state in which the mounting assembly 1700 is not mounted on the motor pack 1600. From another viewpoint, the magnet carrier 1625 may position the magnet 1623 close to the magnetic sensor 1622. The magnet 1623 may be spaced apart from the magnetic sensor 1622 while the mounting assembly 1700 is in a state of being mounted on the motor pack 1600. From another viewpoint, the magnet carrier 1625 may space the magnet 1623 apart from the magnetic sensor 1622.

The magnet carrier 1625 may support the magnet 1623 and limit the range of movement of the magnet 1623. In other words, the position of the magnet 1623 coupled to the magnet carrier 1625 may be limitedly changed by movement or deformation of the magnet carrier 1625.

The magnet carrier 1625 may be accommodated in the sensor housing 1621. The magnet carrier 1625 may be moved within the sensor housing 1621, or the size and shape of the magnet carrier 1625 may be changed within the sensor housing 1621.

In an embodiment, the magnet carrier 1625 may be an elastic member. For example, the elastic member may have a compressible spring shape. The elastic member may be disposed in the sensor housing 1621, with an end portion of the elastic member being fixed and another end of the elastic member being coupled to the magnet 1623. For example, the other end portion of the elastic member may be coupled to the sensor housing 1621, and the other end portion to which the magnet 1623 is coupled may be displaceable without being fixed.

When an external force greater than or equal to a predetermined level is not applied to the magnet 1623, the elastic member may provide an elastic force so that the magnet 1623 is positioned adjacent to the magnetic sensor 1622.

From another viewpoint, the elastic member may maintain an original shape as long as the elastic member does not receive an external force greater than or equal to the predetermined level from the magnet 1623. For example, the magnet 1623 may apply a force to the elastic member due to an attractive or repulsive force acting between the magnet 1623 and the magnetic body or between the magnet 1623 and another magnet 1721. Due to this, the elastic member may be deformed.

Referring again to FIG. 14, the magnet 1623 coupled to the other end portion of the elastic member may be positioned proximal to the magnetic sensor 1622 in a state in which an external force is not applied to the elastic member.

As illustrated in FIG. 15, when the detection target module 1720 is disposed close to the sensor module 1620, the detection target module 1720 may attract the magnet 1623, and thus, the elastic member may be compressed.

For example, the detection target module 1720 may include the magnet 1721 or the magnetic body (not shown) positioned opposite the magnet 1623, and the elastic member may be compressed as the magnet 1721 or the magnetic body of the detection target module 1720 attracts the magnet 1623 of the sensor module 1620.

When a state in which the mounting assembly 1700 is mounted on the mounting part 1612 is defined as a first state and a state in which the mounting assembly 1700 is detached from the mounting part 1612 is defined as a second state, the detection target module 1720 may be closer to the sensor module 1620 in the first state than in the second state.

The magnet 1623 may be spaced apart from the magnetic sensor 1622 in the first state and may be positioned close to the magnetic sensor 1622 in the second state.

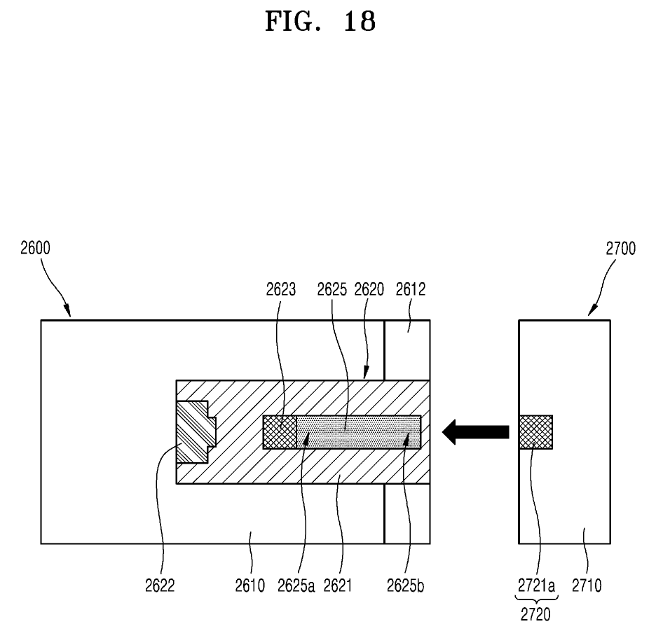

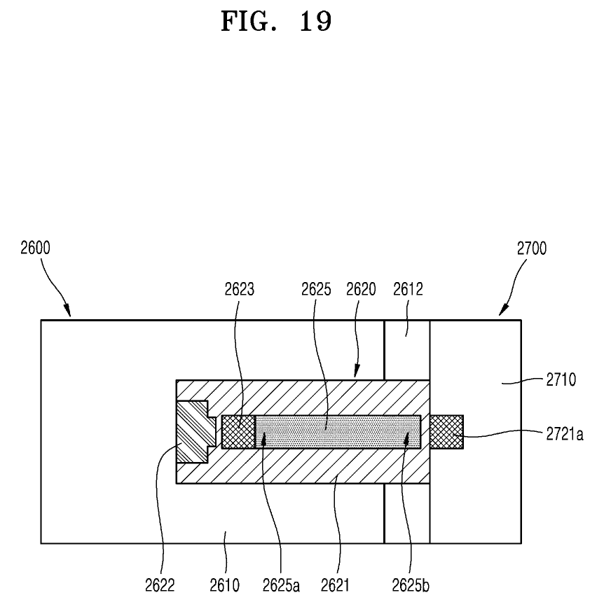

FIGS. 18 and 19 are diagrams schematically illustrating a mounting assembly detection system according to a modification of the second embodiment. Specifically, FIG. 18 is a diagram illustrating a state before a mounting assembly 2700 is mounted on a motor pack 2600, and FIG. 19 is a diagram illustrating a state after the mounting assembly 2700 is mounted on the motor pack 2600.

According to the embodiment illustrated in FIGS. 18 and 19, a magnet 2623 may be spaced apart from a magnetic sensor 2622 in a basic state in which the mounting assembly 2700 is not mounted on the motor pack 2600. From another viewpoint, a magnet carrier 2625 may space the magnet 2623 apart from the magnetic sensor 2622. The magnet 2623 may be disposed proximal to the magnetic sensor 2622 while the mounting assembly 2700 is in a state of being mounted on the motor pack 2600. From another viewpoint, the magnet carrier 2625 may position the magnet 2623 close to the magnetic sensor 2622.

From another viewpoint, the magnet 2623 may be positioned close to the magnetic sensor 2622 in the first state and may be spaced apart from the magnetic sensor 2622 in the second state.

In other words, when an external force greater than or equal to a predetermined level is not applied to the magnet 2623, the elastic member may provide an elastic force so that the magnet 2623 is spaced apart from the magnetic sensor 2622.

Referring again to FIG. 18, the magnet 2623 coupled to the other end portion of the elastic member may be spaced apart from the magnetic sensor 2622 in a state in which an external force is not applied to the elastic member.

As illustrated in FIG. 19, when the detection target module 2720 is disposed close to the sensor module 2620, the detection target module 2720 may push the magnet 2623, and thus, the elastic member may be tensioned.

The detection target module 2720 may include a magnet 2721a disposed opposite to the magnet 2623, and the elastic member may be tensioned as the magnet 2721a of the detection target module 2720 pushes the magnet 2623 of the sensor module.

In another embodiment, the magnet carrier 2625 may include a support (not shown) which supports the magnet 2623, and an elastic member (not shown). The elastic member may be coupled to the support so that the support member is movable according to deformation of the elastic member. For example, the support may be rotatable around an axis. A rotation shaft may be inserted into an end portion of the support, and a magnet may be coupled to another end portion of the support. The elastic member may be coupled to an area of the support so that the support is rotatable as the elastic member is compressed or tensioned.

However, the concept of the disclosure is not limited thereto, and the magnet carrier 2625 may adopt various structures in which the magnet 2623 is movable according to deformation of the elastic member.

As described above, the magnet 2623 is not in a fixed position, and a distance between the magnet 2623 and the magnetic sensor 2622 may change depending on deformation of the elastic member. Therefore, when the change in magnetic field occurs due to the movement of the magnet 2623, the magnetic sensor 2622 may detect the change in magnetic field and the mounting assembly detection system may determine whether the mounting assembly is mounted through the sensor module 2620.

According to embodiments, the mounting assembly may be sensed without externally exposed detection pins or the like. Therefore, the mounting assembly detection system according to the embodiment may have no concern that the pin is damaged, unlike the method in which the mounting assembly presses the protruding pin, and may solve a recognition error which may occur when the pin is pressed incorrectly.

According to the disclosure, a mounting assembly detection system capable of minimizing damage caused by contact or pressure from an external object may be provided.

The disclosure has been described with reference to some embodiments. Those of ordinary skill in the art will understand that the disclosure may be implemented in modified forms without departing from the essential features of the disclosure. Therefore, the disclosed embodiments should be considered in an illustrative sense rather than a restrictive sense. The scope of the disclosure is indicated in the claims rather than the foregoing description, and all differences within the scope equivalent thereto should be construed as falling within the disclosure.

What is claimed is:

1. A mounting assembly detection system comprising:
a motor pack configured to provide a driving force to a surgical instrument; and
a mounting assembly including at least one detection target module, and configured to be detachably mounted on the motor pack,
wherein the motor pack comprises at least one sensor module disposed adjacent to a mounting part configured to provide an area in which the mounting assembly is mounted,
wherein the at least one sensor module comprises:
a first magnetic sensor; and
a first magnetic body having a first area adjacent to the first magnetic sensor and a second area adjacent to the mounting part, and
wherein the at least one detection target module comprises a first magnet disposed at a position corresponding to the at least one sensor module.

2. The mounting assembly detection system of claim 1, wherein the first magnet is disposed at a position corresponding to the second area of the first magnetic body.

3. The mounting assembly detection system of claim 1, wherein
the mounting assembly detection system includes a first state in which the mounting assembly is mounted on the mounting part and a second state in which the mounting assembly is detached from the mounting part, and
the at least one detection target module is closer to the at least one sensor module in the first state than in the second state.

4. The mounting assembly detection system of claim 3, wherein
the first magnetic body is magnetized by the first magnet in the first state, and
the first magnetic sensor is configured to detect the at least one detection target module when the first magnetic body is magnetized.

5. The mounting assembly detection system of claim 1, wherein the first magnetic sensor is spaced apart from the first magnet by a first preset distance while the mounting assembly is in a state of being mounted on the motor pack.

6. The mounting assembly detection system of claim 1, wherein the first magnetic body is spaced apart from the first magnet by a second preset distance while the mounting assembly is in a state of being mounted on the motor pack.

7. The mounting assembly detection system of claim 1, wherein the at least one sensor module further comprises a sensor housing configured to accommodate the first magnetic sensor and the first magnetic body, and
wherein at least a portion of the sensor housing is coupled to the mounting part.

8. The mounting assembly detection system of claim 1, wherein the at least one sensor module of the motor pack includes a plurality of sensor modules, and the at least one detection target module of the mounting assembly includes a plurality of detection target modules corresponding to the plurality of sensor modules.

9. The mounting assembly detection system of claim 8, wherein the mounting assembly detection system is configured to detect the mounting assembly when the plurality of sensor modules detect all of the plurality of detection target modules.

10. The mounting assembly detection system of claim 1,
wherein the motor pack further comprises:
a second magnetic sensor; and
a second magnetic body having a first area adjacent to the second magnetic sensor and a second area adjacent to the mounting part, and
wherein the mounting assembly further comprises a bridge module including a third magnetic body disposed at a position corresponding to the at least one sensor module.

11. The mounting assembly detection system of claim 10, wherein, when the mounting assembly is an adapter disposed between the surgical instrument and the motor pack, the third magnetic body and the second magnetic body are magnetized by a second magnet provided in the surgical instrument while the adapter and the surgical instrument are in a state of being mounted on the motor pack.

12. A mounting assembly detection system comprising:
a motor pack configured to provide a driving force to a surgical instrument; and
a mounting assembly including a detection target module, and configured to be detachably mounted on the motor pack,
wherein the motor pack comprises a sensor module disposed adjacent to a mounting part configured to provide an area in which the mounting assembly is mounted,
wherein the sensor module comprises:
a magnetic sensor;
a first magnet configured to be movable between a position adjacent to the magnetic sensor and a position spaced apart from the magnetic sensor; and
a magnet carrier configured to support the first magnet and limit a range of movement of the first magnet, and
wherein the detection target module is disposed at a position corresponding to the sensor module.

13. The mounting assembly detection system of claim 12, wherein the detection target module comprises a second magnet or a magnetic body disposed to face the first magnet.

14. The mounting assembly detection system of claim 12, wherein
the mounting assembly detection system includes a first state in which the mounting assembly is mounted on the mounting part and a second state in which the mounting assembly is detached from the mounting part, and
the detection target module is closer to the sensor module in the first state than in the second state.

15. The mounting assembly detection system of claim 14, wherein the magnet carrier comprises an elastic member configured to provide an elastic force to bring the first magnet proximal to the magnetic sensor when an external force greater than or equal to a predetermined level is not applied to the first magnet.

16. The mounting assembly detection system of claim 15, wherein the first magnet is spaced apart from the magnetic sensor in the first state and positioned proximal to the magnetic sensor in the second state.

17. The mounting assembly detection system of claim 14, wherein the magnet carrier comprises an elastic member configured to provide an elastic force to cause the first magnet to be spaced apart from the magnetic sensor when an external force greater than or equal to a predetermined level is not applied to the first magnet.

18. The mounting assembly detection system of claim 17, wherein the first magnet is positioned proximal to the magnetic sensor in the first state and spaced apart from the magnetic sensor in the second state.

19. A motor pack configured to provide a driving force to a surgical instrument, the motor pack comprising:
a mounting part configured to support an adapter comprising a detection target module; and
a sensor module disposed adjacent to the mounting part,
wherein the sensor module comprises:
a magnetic sensor; and
a magnetic body having a first area adjacent to the magnetic sensor and a second area adjacent to the mounting part.

20. The motor pack of claim 19, wherein the detection target module comprises a magnet arranged so as to correspond to the magnetic body.
